(19) United States
(12) Patent Application Publication
(10) Pub. No.: US 2011/0264151 A1
(43) Pub. Date: Oct. 27, 2011

(54) BONE FIXATION DEVICE AND METHOD OF VALIDATING ITS PROPER PLACEMENT

(76) Inventors: TIMOTHY DAVIS, SANTA MONICA, CA (US); HYUN W. BAE, LOS ANGELES, CA (US)

(21) Appl. No.: 13/093,060

(22) Filed: Apr. 25, 2011

Related U.S. Application Data
(60) Provisional application No. 61/327,901, filed on Apr. 26, 2010.

(51) Int. Cl. A61B 17/86

(52) U.S. Cl. 606/305

(57) ABSTRACT

A bone fixation device, in particular a pedicle screw, is provided. The device includes an enhanced bone-to-metal interface, while also having electroconductive properties. Also provided are methods of using the bone fixation device and methods of validating proper placement of the device.

### CROSS-REFERENCE TO RELATED APPLICATIONS

This application claims priority to U.S. Provisional Application No. 61/327,901 filed on Apr. 26, 2010 of the same title, the entire contents of which are incorporated by reference.

### FIELD

The present disclosure relates to implantable devices for the surgical treatment or repair of damaged or compromised bone, and more particularly to a mechanical fixation device for stabilizing or immobilizing a weakened, injured or fractured segment of bone, and methods for using such a device and for validating proper alignment or placement of the device.

### BACKGROUND

There are a number of reasons that can cause bone to become damaged or injured. For example, bone can become fractured as a result of external physical trauma or force. Bone can erode or become brittle due to osteoporosis or other degenerative diseases. Or, bone can weaken or become unstable due to osteoarthritis or over time with the natural aging process. Where the bone injury is not able to heal itself through the body's natural repair process, or in cases of severe bone loss or damage, surgical intervention may be required and may involve the insertion of a fixation device to replace, repair or immobilize the damaged tissue.

For some spinal injuries, it may be desirable to use bone fixation devices such as pedicle screws as part of a rigid construct to stabilize and, if needed, promote fusion of the bone. The technique of posterior instrumented spinal fusion is well known, along with the use of pedicle screws for posterior fixation. Currently, pedicle screw fixation is applied to a variety of spinal pathologies. The pedicle screws are most commonly implanted under fluoroscopic guidance, though there are many other known techniques for pedicle screw placement.

One of the most common complications in posterior spinal fixation is undetected screw misplacement, leading to breach of the pedicular wall and possibly encroachment or penetration of neural tissue causing neural deficits. Cortical breach by the pedicle screw can lead to postoperative pain or parasthesias in mild cases, and even paralysis in the most severe situations. Hence, avoiding these types of complications is of utmost importance.

One known assistive technique for verifying screw placement is triggered electromyography (tEMG). Along with intraoperative imaging, the use of tEMG testing has been known for confirming proper placement of pedicle screws. tEMG can be utilized prior to, or after, screw placement to assist with the detection of pedicle breach. The technique involves the use of a monopolar probe to deliver a current to the head of the pedicle screw which acts as an extension of the stimulating probe into deeper tissues. The electrical current is carried into the bone and soft tissues surrounding the pedicle screw. If the pedicle screw is adjacent to neural structures due to pedicle breach, then the current would flow along the neural tissue to the end organ muscle and will produce an electromyographic response. Electrodes placed in corresponding myotomes can pick up the tEMG activity.

Generally, an electromyographic response can occur at lower stimulation thresholds if the pedicle screw has breached the pedicle wall. A stimulation threshold is determined as being the minimum current necessary applied to the pedicle screw to evoke an electromyographic response. Previous studies have shown that medial or inferior cortical breach is associated with stimulation thresholds of less than 9 mA. Thresholds of 10 mA or greater usually indicate a screw is placed well within the confines of the cortices of the pedicle.

In any electrical circuit, current requirements will change depending on the electroconductive properties of the parts of that circuit. Therefore, if resistance levels of pedicle screws vary significantly amongst types or vendors, the tEMG thresholds may be inconsistent. A pedicle screw with a higher electroconductive resistance would produce current thresholds higher than expected. This elevated stimulation threshold could be interpreted as indicating proper screw placement when in fact the pedicle screw could be misplaced. This would result in a false-negative conclusion and may result in post procedure complications.

The resistive value of a pedicle screw may vary depending on the material from which it is constructed. Titanium, stainless steel, and other metals used for medical implants have relatively low electrical resistance and are considered good electrical conductors by International Annealed Copper Standard (IACS). Materials that possess high electrical resistance require higher levels of electrical current in order to conduct a set amount of electricity through a set volume or length of material. Materials that do not conduct electricity due to exponentially higher electrical resistance are considered insulators and cannot be measured on an IACS chart. Previous studies have shown that screws constructed from stainless steel have resistive properties nearly identical to titanium pedicle screws.

To improve the performance of the pedicle screws, many use hydroxyapatite (HA) as a coating for the pedicle screw shank to promote bone in-growth and increase “pullout” strength. HA is a naturally occurring mineral form of calcium apatite. Because of its porous molecular structure, this ceramic improves bone-to-metal interface on orthopedic devices such as pedicle screws. However, as applicants have observed and confirmed, HA has a high electrical resistance value (i.e., is a poor conductor of electrical current) and, in fact, acts similar to other insulators like rubber, glass, and porcelain. Accordingly, HA coated pedicle screws, while providing an improved bone-to-metal interface and therefore better mechanical stability, would not be able to conduct electrical current and is thus incompatible with tEMG technology. In fact, manufacturers of HA coated pedicle screws have warned of inconsistent stimulation thresholds during tEMG testing. Electrical current applied to an HA coated pedicle screw would travel through the metal shank, but would not conduct through the HA due to its high insulative properties. Therefore, any stimulation thresholds would not only be inconsistent, but falsely high even in the case of a pedicle breach or contact with neural tissue.

Accordingly, it is desirable to provide a bone fixation device, or more specifically a pedicle screw, that can offer the benefits of an improved bone-to-metal interface while also being suitable for use with tEMG assistive techniques or other alternative electrical monitoring techniques to identify and detect improper positioning so as to avoid pedicle breach of the screw during its placement. It is further desirable that such a device, along with the method of its use, offers an accurate, easy to use (i.e., simple to administer) and efficient (i.e., doesn't require longer operating room (OR) time for the patient) manner for the surgeon to achieve this goal.

### SUMMARY

The present disclosure provides a bone fixation device, and more specifically a pedicle screw, that includes an enhanced bone-to-metal interface such as a hydroxyapatite coating while also being electroconductive. Also provided are methods of using the bone fixation device and validating proper placement of the device.

In one exemplary embodiment, a bone fixation device is provided. The device comprises a bone fastener having a first, leading end and a second, trailing end, and an elongate shaft extending therebetween. The shaft can have external threads with cutting edges and have a coating for promoting bone growth. The device also comprises a head component having a pair of sidewalls defining a slot in between, the sidewalls extending from a base portion, the base portion also including an opening for receiving the bone fastener. In addition, a portion of the shaft comprises exposed metal and the device is electroconductive.

In another exemplary embodiment, a method of validating proper placement of a bone fixation device is provided. The method includes the steps of providing a bone fixation device having a bone fastener having a first, leading end and a second, trailing end, and an elongate shaft extending therebetween, the shaft having external threads with cutting edges and a coating for promoting bone growth, and a head component having a pair of sidewalls defining a slot in between, the sidewalls extending from a base portion, the base portion also including an opening for receiving the bone fastener, wherein a portion of the shaft comprises exposed metal and the device is electroconductive. The device is placed into a bone, and electrical stimulus is applied to the device. An electrical response is measured. Finally, based on the value of the measured response, it can be determined whether the device is positioned properly in the bone.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a perspective view of an exemplary embodiment of a bone fixation device of the present disclosure.

FIG. 2 is a side view of the bone fixation device of FIG. 1.

FIG. 3 is an enlarged view of a portion of the bone fixation device of FIG. 2.

FIG. 4 illustrates an example of proper positioning of the bone fixation device of FIG. 1 and improper positioning in situ.

FIG. 5 represents the manner of electric current flow through each of the bone fixation devices of FIG. 4.

FIG. 6A is a partial cutaway view of another exemplary embodiment of a bone fixation device of the present disclosure.

FIG. 6B is a partial cutaway view of yet another exemplary embodiment of a bone fixation device of the present disclosure.

### DESCRIPTION OF THE EMBODIMENTS

In general, the present disclosure provides a bone fixation device, and more specifically a pedicle screw, that advantageously possesses the dual properties of an enhanced bone-to-metal interface, such as with a hydroxyapatite (HA) coating or other coating that promotes bone ingrowth, while also being electroconductive. This device would allow for improved bone growth, but maintain the electroconductivity that is necessary for the utilization of triggered electromyographic testing (tEMG) to confirm proper screw placement.

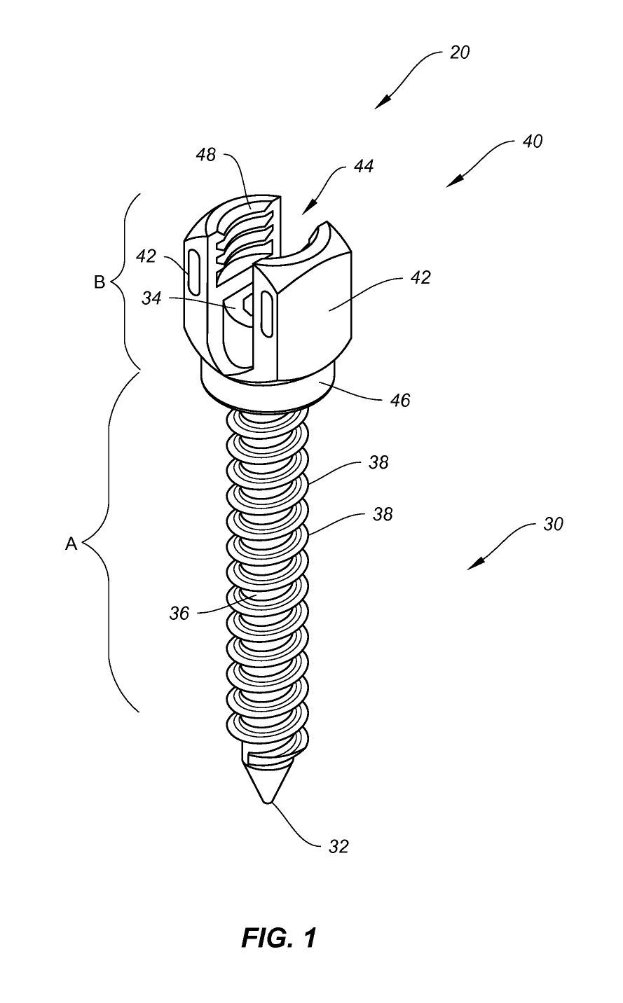

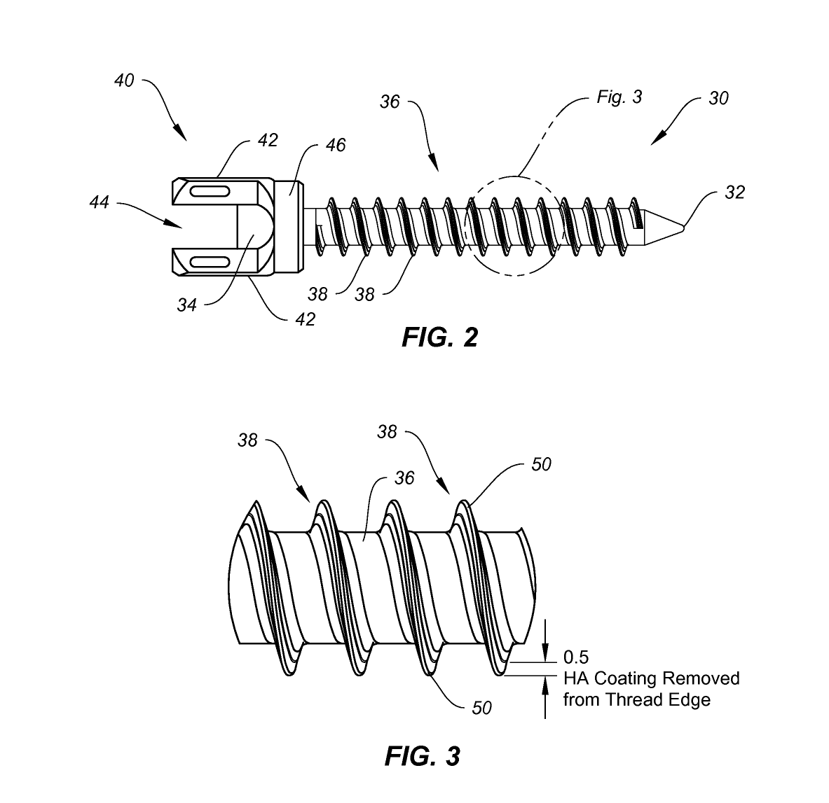

FIGS. 1 and 2 show an exemplary embodiment of a bone fixation device 20 of the present disclosure. The device 20 comprises two main components: the A portion comprises the anchoring element that is configured to be inserted into bone, while the B portion comprises the head element that is configured to receive another device, usually an implantable rod. The A portion, or the anchoring element, may include a fastener or screw 30 having a first, leading end 32 and a second, trailing end 34. An elongate shaft 26 may extend in between the ends 32, 34. The shaft 36 may also include external threads 38. The first, leading end 32 may be configured as a tapered tip. However, it is contemplated that the leading end 32 may have other configurations. For example, the leading end 32 may also be blunt if the screw 30 is inserted into a pre-drilled hole. Alternatively, the leading end 32 may be fashioned with a self-drilling or self-tapping end, depending on the needs of the surgeon.

The B portion of the device 20 comprises a head element 40 that can include a pair of sidewalls 42 defining in between a slot 44. In some applications, the slot 44 may be configured to receive another component, such as a rod, for example. At the base 46 where the sidewalls 42 meet is an opening for receiving the A portion. The A portion of the device 20 can be inserted into the opening of the base 46, where the second, trailing end 34 can reside. It is contemplated that the device 20 of the present disclosure may be monoaxial or polyaxial. Sidewalls 42 may also include internal threads 48, as shown. While not shown here, the device 20 may also include a closure element that is configured to mate with the head element 40 and close off the slot 44. The closure element could be a locking cap having a threaded shaft, for example.

The device 20 itself and its components may be formed of any suitable biocompatible metal as is known in the art, such as for example, stainless steel, titanium or titanium alloy. Additionally, the shaft 36 and portions of the threads 38 of the screw 30 may be coated with hydroxyapatite or other material that promotes bone ingrowth and improves the bone-to-metal interface. Hydroxyapatite, or HA, is a naturally occurring ceramic which has been used as a coating on orthopedic implants to improve bone-to-metal interface by promoting bone ingrowth and incorporation. Accordingly, the inclusion of HA on the screw 30 provides the benefits of enhanced stability of the device 20 after placement into bone.

However, a portion of the coating is absent from the outer edge 50 of the threads 38, as shown in greater detail in FIG. 3, which represents an enlarged view of the encircled portion of FIG. 2. This portion can correspond to about the midsection of the screw 30, or range from about the second-third to lower-third of the screw 30. As applicants have discovered, HA acts like an insulator, serving as a poor electrical conductor. Exposure of metal would allow for some electricity to be conducted, so that the device 20 can be placed with assistance from triggered electromyography. Alternatively, the screw 30 may have multiple, varying coatings having different electrical conductive properties. For example, in addition to the HA coating, the outer edge 50 of the threads 38 may be coated with a coating that is electrically conductive.

In one embodiment, the midsection of the shaft 36, as designated by the circled section in FIG. 2, can be configured to conduct electricity. The threads 38 within Section A would have no HA coating (i.e., metal is exposed) at the threads' cutting edge 50, and thus not impede electrical current. The outer cutting edge 50 of the bone screw 30, being the largest point of radius from center, would be the most likely portion of the screw 30 to penetrate through the pedicle wall first, thereby causing breach. In other words, the exposed metal on the threads' edges act like a strip of metal along the outer most cutting edge of the threads 38 in Section A, allowing for electrical current to conduct through this area of the screw 30 that would potentially breach the pedicle wall. The exposed metal makes the device 20 of the present disclosure suitable for placement using triggered electromyography techniques, or other alternative electric monitoring assistance techniques.

Pedicle breach can be, to variable degrees, full or partial breach. In either scenario, the first part of the screw 30 to breach would be the leading edge 50 of the threads 38. tEMG testing is usually applied once the screw 30 is placed fully into the vertebrae. In this example, the distal third (i.e., tip region 32) of the screw 30 would be buried in the body of the vertebrae and would be insulated from neural tissue. An approximately one to three millimeter strip of exposed metal, free of HA coating, at the outer edge 50 of the threads 38 of the middle to proximal thirds of the screw 30 would potentially be in contact with neural tissue in a pedicle breach. The outer edge 50 of the threads 38 would serve as the location of exposed metal through which electrical current can conduct, thereby making the screw 30 compatible with the use of tEMG.

In one embodiment, a method for creating the screw 30 of the present disclosure may first utilize standard techniques for coating the shaft 36 and threads 38 with hydroxyapatite. Then, the threads 38 in one portion of the screw 30, such as the midsection roughly corresponding to the area encircled in FIG. 2, may be milled clean to leave an exposed metal strip along the outer cutting edge 50 of the threads 38 in this section.

In another embodiment, a method for creating the screw 30 of the present disclosure may contain a step of preventing binding of the hydroxyapatite to the metal of the cutting edge 50 of some of the threads 38 during the process of coating the screw 30. For example, it is contemplated that a removable mask may be applied to selected portions of the threads 38 to prevent HA from binding to the area under the mask. After coating, the mask may be removed to provide exposed metal.

In yet another embodiment, a method for creating the screw 30 of the present disclosure may involve the application of a removable coating along the outer cutting edge 50 of the threads 38 that binds with the HA. After HA coating, the removable coating with the HA can be taken off, leaving a bare strip of exposed metal along the outer cutting edge 50 of the threads 38.

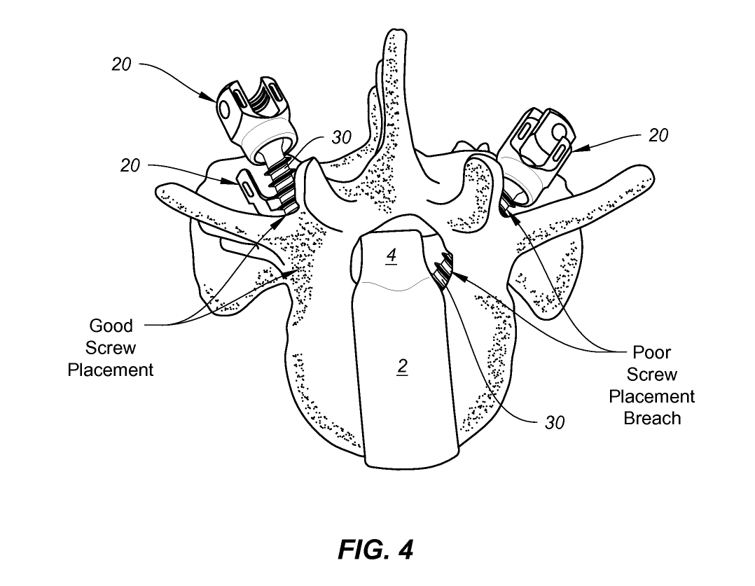

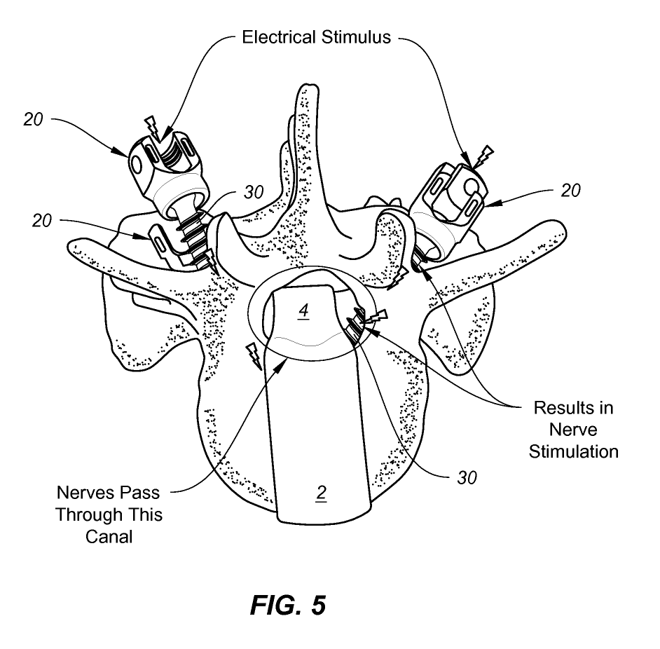

FIGS. 4 and 5 represent one contemplated method of using the device 20, in which the device 20 can be placed into a pedicle of a vertebra 2 while utilizing triggered electromyography, as a means to validate proper placement of the device 20. As shown, two properly positioned devices 20 are provided on the right side of the vertebral column. These devices 20 show good screw 30 placement into the pedicle of the vertebral body 2, with no breach. On the left side of the same vertebral column, a similar device 20 has been positioned but improperly. As the arrowed label indicates, the screw 30 has breached the pedicle wall, and portions of the midsection of the shaft 36 are exposed within the canal 4.

FIG. 5 represents one method of validating proper placement of the device 20 in situ. As shown, triggered electromyography techniques may be applied to each of the implanted devices 20. In the properly positioned devices 20 on the right side of the vertebral column, no electromyographic response is expected after triggering. Conversely, the malpositioned device 20 on the left side of the vertebral column is expected to generate an electromyographic response when triggered, as the threshold to produce one is lower in the breached device due to nerve stimulation in the canal 4.

Although the device 20 shown and described herein comprises two components A and B forming a pedicle screw, it is contemplated that the device 20 could also be configured as a unitary body for use without a rod or other rigid construct.

It is contemplated that the amount of threads 38 coated with the bone-to-metal enhancement versus the amount of threads 38 exposed may vary depending on the amount of conductive area that is desired while balancing the desired effect of the bone-to-metal coating. In addition, the configuration in which the threads 38 are exposed may vary along the length of the shaft 36. For example, the threads 38 closer to leading end 32 may be more exposed relative to other threads 38 further away from the leading end 32. Indeed, the threads 38 near leading end 32 may be completely exposed, while the other threads 38 along shaft 36 may have progressively more HA coating. Alternatively, the outer edge 50 of all or some of the threads 38 may have at least some exposed portions. Other variations are consistent with the principles of the present invention, and are described below.

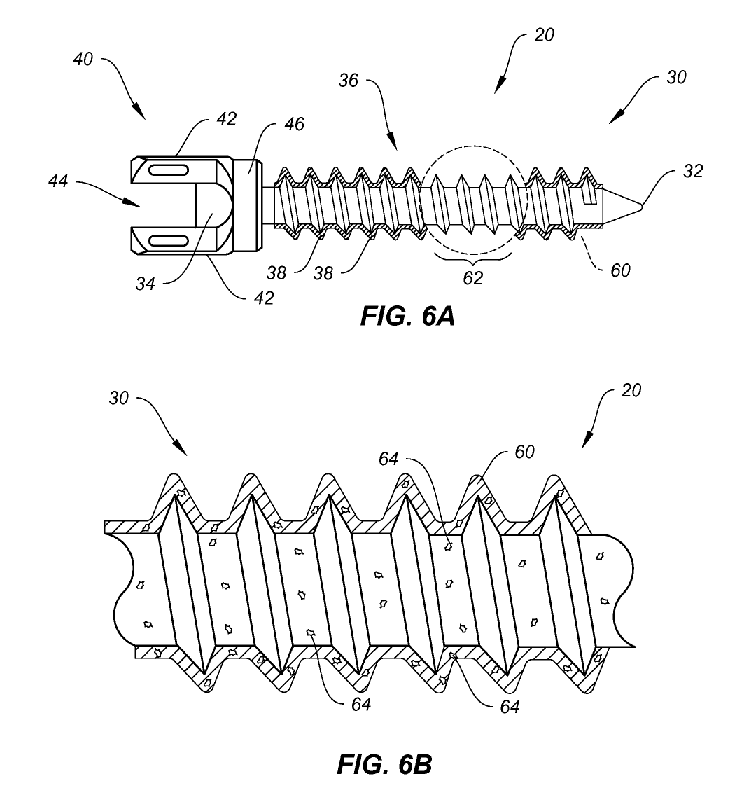

FIG. 6A illustrates another exemplary embodiment of a bone fixation device 20 of the present disclosure that shares similar features to the bone fixation device of FIG. 1, with like elements being represented by similar reference numerals. In the present embodiment, the bone screw 30 may have a partial coating 60 of a bone-to-metal enhancement such as a ceramic like HA. Exposed, however, is a portion 62 of the screw 30 approximately at the encircled region shown. This portion 62 corresponds to the area of the screw most likely to be exposed in a pedicle wall breach, and is roughly around the midsection or ranging from the second-third to the lower third of the screw 30.

FIG. 6B illustrates still another exemplary embodiment of a bone fixation device 20 of the present disclosure that shares similar features to the bone fixation device of FIG. 1, with like elements being represented by similar reference numerals. In the present embodiment, the screw 30 is completely coated with the bone-to-metal enhancement. The coating 60, however, could contain embedded metal flakes or beads 64, in such a quantity and in a configuration that allows electroconductivity without sacrificing the benefits of the enhanced bone ingrowth properties of the coating. For instance, the metal flakes or beads 64 could be configured to create a continuous metal webbing or net that surrounds the shaft 36 and threads 38. This metallic webbing or net would allow for electroconductivity without significantly impeding screw threading or bone ingrowth.

Finally, as previously mentioned, in other contemplated embodiments the screw 30 may have multiple, varying coatings whereby the coatings can have different electrical conductive properties. For example, in addition to an HA coating, the outer edge 50 of the threads 38 may be coated with a coating that is electrically conductive.

Although the devices shown and described herein utilize HA as an example of a bone growth promoting substance that provides an enhanced bone-to-metal interface, it is understood that other alternative bone growth promoting coatings or substances may just as easily be used in accordance with the principles of the disclosure. These alternatives include, for example, nano-apatite coatings, hyaluronic acid, and protein coatings, with or without growth factors.

While the invention has been described in detail and with reference to specific examples therein, it will be apparent to one skilled in the art that various changes and modifications can be made without departing from the spirit and scope thereof.

What is claimed is:

1. A bone fixation device, comprising:
a bone fastener having a first, leading end and a second, trailing end, and an elongate shaft extending therebetween, the shaft having external threads with cutting edges and further including a coating for promoting bone growth; and
a head component having a pair of sidewalls defining a slot in between, the sidewalls extending from a base portion, the base portion also including an opening for receiving the bone fastener;
wherein a portion of the shaft comprises exposed metal, and the device is electroconductive.

2. The device of claim 1, wherein the coating is resistant to electroconductivity.

3. The device of claim 2, wherein the coating is a ceramic coating.

4. The device of claim 3, wherein the coating is a hydroxyapatite coating.

5. The device of claim 1, wherein the coating covers more than two-thirds of the external threads of the shaft.

6. The device of claim 7, wherein the coating is discontinuous.

7. The device of claim 6, wherein some of the external threads have exposed cutting edges with no coating thereon.

8. The device of claim 7, wherein the exposed cutting edges are located around a midsection of the shaft.

9. The device of claim 6, wherein all of the external threads have at least some coating thereon.

10. The device of claim 1, wherein the coating includes an electroconductive element.

11. The device of claim 10, wherein the electroconductive element comprises a metal flake or bead.

12. The device of claim 10, wherein the electroconductive element forms a metallic web around the shaft.

13. The device of claim 1, further including a second coating, wherein the second coating is more electroconductive than the first coating.

14. The device of claim 1, further being configured for placement using triggered electromyography assistance.

15. A method of validating proper placement of a bone fixation device, comprising the steps of:
providing a bone fixation device having a bone fastener having a first, leading end and a second, trailing end, and an elongate shaft extending therebetween, the shaft having external threads with cutting edges and further including a coating for promoting bone growth; and a head component having a pair of sidewalls defining a slot in between, the sidewalls extending from a base portion, the base portion also including an opening for receiving the bone fastener, wherein a portion of the shaft comprises exposed metal, and the device is electroconductive;
placing the device into a bone;
applying an electrical stimulus to the device;
measuring an electrical response of the device; and
determining if the device is positioned properly in the bone.

16. The method of claim 15, wherein the bone is a vertebral body and the step of placing includes inserting the device into a pedicle.

17. The method of claim 16, wherein the step of determining includes determining if the device has breached into the canal of the vertebral body.

18. The method of claim 16, wherein the step of applying includes applying an electrically conducting probe to the device.

19. The method of claim 16, wherein the electrical response is an electromyographic response.

20. The method of claim 16, wherein the step of determining includes comparing the value of the measured electrical response to a threshold value.
